(19) United States
(12) Patent Application Publication
(10) Pub. No.: US 2018/0315442 A1
(43) Pub. Date: Nov. 1, 2018

(54) PERSONALIZED INSTANT MOOD IDENTIFICATION METHOD AND SYSTEM

(71) Applicant: Smartmedical Corp., Tokyo (JP)

(72) Inventor: Takaaki Shimoji, Tokyo (JP)

(21) Appl. No.: 16/029,633

(22) Filed: Jul. 8, 2018

Related U.S. Application Data
(63) Continuation of application No. 14/701,527, filed on May 1, 2015, now abandoned.

(51) Int. Cl. G10L 25/63; G09B 19/00; G09B 5/06; G09B 5/02; A61B 5/16; A61B 5/01; A61B 5/08; A61B 5/024; A61B 5/021; A61B 5/00

(52) U.S. Cl. CPC G10L 25/63; G09B 19/00; G09B 5/06; G09B 5/02; A61B 5/7203; A61B 5/01; A61B 5/0816; A61B 5/024; A61B 5/021; A61B 5/165

(57) ABSTRACT

Methods and systems of identifying an instant mood state personalized for an individual subject are described. The methods and systems may include analysis of biological information specific to the subject, such as voice information acquired through speech, to provide information relating to an emotional state factor of the subject. Emotional state factor information may be used in conjunction with a decision means such as a database or decision tree relating the emotional state factor to certain moods personalizable for the individual subject. The decision means may be expandable, changeable, and/or capable of incorporating information self-reported by the subject to refine and optimize the information therein relating measured emotional state factors to specific moods. Identified instant mood states may be employed by the individual user in day to day life, and may also be employed by others providing care for, or a service to, the individual user.

### CROSS-REFERENCE TO RELATED APPLICATION

This application is a continuation of U.S. patent application Ser. No. 14/701,527, filed 1 May 2014, which is incorporated herein by reference in its entirety.

### TECHNICAL FIELD

This disclosure generally relates to the field of methods for identifying a mood, and to systems for identifying a mood. In particular, this disclosure relates to a method for identifying an instant mood that may be tuned to an individual subject using at least one type of biological information from the subject, and to a system for identifying an instant mood tuned to an individual subject using at least one type of biological information from the subject.

### BACKGROUND

Humans may undergo a variety of changing emotional states throughout a given day. These emotional states are often transient in nature, and may be caused by exposure to a sudden stimulus. Emotional states may arise and dissipate over a short period of time in response to an immediate circumstance, including aural, visual, or physical stimuli; essentially stimuli encased in the words and/or actions of another. The words and/or actions may be immediate in nature, occurring in the vicinity of the human experiencing the emotion, and may also be caused by learning about something that has taken place a great distance away.

Moods, on the other hand, may last for a longer period of time, typically at least one or two days, be less intense than emotions, and not exhibit a single stimulus or cause. Moods may relate to cumulative changes in emotional states over a given period of time, and may be entrenched and difficult to voluntarily change, even with perseverance. Emotional states are typically sharper and more varied than moods that are influenced by different emotions, and humans may be able to identify several different specific emotional states they may currently be experiencing. Moods may be understood in more generalized terms. When asked about their current mood, people may answer in vague terms such as good mood or bad mood.

Emotional states can be experienced at the same time as moods, but they may be more immediate and visible than moods. Examples include sudden feelings of happiness or joy while under the influence of a bad mood, as well as sadness or anger while in a good mood. A mood may be able to influence emotional states so that the emotional state approaches the mood. While it may appear that emotions are dominant to moods due to their immediacy and strength, emotions may also be susceptible to mood, which could make it more likely for a person to interpret his or her environment in particular ways, even distorting the person's interpretation of a particular situation. When in a bad mood, for example, it may be much easier to misinterpret even positive experiences or emotions in the light of the bad mood.

It may be advantageous for a subject to have clear knowledge of the mood the person is experiencing at a current time. Such moods may be characterized as an “instant mood”, which may be found by analyzing current emotional states, or a “non-instant” mood, which may be found by weighing current emotional states with instant mood information for a predefined period of time prior to the current time. An instant mood may be determined from distinct human emotional states the subject is currently experiencing by weighing and/or combining at least three or more of the emotional states. Although an identified instant mood may exhibit more variability over a short term than a non-instant mood, which is discussed above, instant mood information may be beneficial for the subject in comparison to referencing simple emotional state levels.

Understanding the similarities and differences between instant moods, non-instant moods, and emotions, and particularly their differences, may require time and practice. Such understanding may influence public acceptance of methods and systems of identifying personalized emotional states and moods. Learning that the anger and frustration a subject may be feeling isn't caused by others in the immediate vicinity, but instead by a non-instant mood that the person has been feeling before, may be beneficial. The person may then be able to accept that people in the immediate vicinity are not the cause of a specific emotional reaction. Alternatively, a series of poor instant moods may be able to be remedied when explicitly known by a user or user's caregiver.

Conventional measurement techniques exist for identifying emotional states. U.S. Pat. No. 7,340,393 discloses a method for detecting emotion utilizing the voice of a subject using intensity, tempo, and intensity characteristics found in voice input, for example. Intensity change patterns are computed for each of the characteristics, and then compared to predefined values to identify emotional states including anger, sadness, and pleasure. The predefined values may be averages or other computed values obtained from measured data of multiple subjects, and may be stored in a database.

U.S. Pat. No. 7,340,393 does not, however, disclose utilizing the emotional states to identify personalized instant moods or non-instant moods in a subject. Further, comparisons are made between computed values from voice data, and predefined values that may not be suitable or optimized for a range of individual subjects, but rather only for an average subject.

It may be advantageous to provide a personalized mood identification method and system capable of identifying an instant mood of a subject based on computed values found using at least one type of biological information from the subject, and optionally comparing the computed values with values specifically fine-tuned through an interactive learning process to the individual subject in order to determine end emotional states and/or identify a personalized instant mood for the subject. Moods, instant and non-instant, may be identified from a weighted combination of emotional states, as well as by weighing a current state found from the emotional states to stored information from the subject over a fixed period of time, such as over the past several hours or days. Emotional states and moods thus identified may be more useful to the subject than similar computations made using average values found from a large population of test subjects.

### SUMMARY

This disclosure relates to a method and a system of identifying an instant mood of a subject. At least one type of biological information unique to the subject may be used, and an emotional state may be identified in addition to an instant mood. When voice information is used as the biological information, a personal electronic device such as a mobile phone or smart phone may be advantageously employed to input the biological information. Through a serious of repeated learning opportunities, the identified instant mood may be tuned or optimized for the individual subject, thus identifying a personalized instant mood unique to the subject and differing from similar computations made for different subjects.

At least one embodiment may be summarized as a method of identifying an instant mood of a subject, including inputting a signal that includes at least one type of biological information from a subject; computing, using a first microprocessor, at least one characteristic numerical value from the signal; computing, using a second microprocessor, at least one emotion factor from the at least one characteristic numerical value; comparing the emotion factor with at least one entry of a predefined decision means to identify an instant mood; and providing the instant mood to the subject.

The first microprocessor and the second microprocessor may be the same microprocessor.

The method of identifying an instant mood of a subject may further include providing the at least one characteristic numerical value to a second microprocessor.

The predefined decision means may be refined for the subject being tested.

The method of identifying an instant mood of a subject may further include: querying the subject as to the correctness of the provided instant mood; receiving feedback information from the subject concerning the correctness of the provided instant mood; and using the feedback information to refine the decision means to reflect the feedback information from the subject.

The at least one type of biological information may include voice information, and the voice information may include a unit of spoken speech.

The emotion factor may represent at least three distinct human emotions, and the three distinct human emotions may be anger, sadness, and happiness.

The identified instant mood may be stored in anon-volatile storing means, and the instant mood may be computationally weighted against at least one previously identified instant mood stored in the non-volatile storing means.

The voice information may be input using a personal electronic device including the first microprocessor.

The personal electronic device may be selected from the group consisting of mobile phones, smart phones, tablet computers, and mobile media playing devices.

The second microprocessor may be located remotely from the subject, and at least one of the second microprocessor and the non-volatile storing means may be located in a cloud computing infrastructure.

The voice information may undergo noise cancellation.

The instant mood may be provided to the subject using a personal electronic device including the first microprocessor, and the personal electronic device may be selected from the group consisting of mobile phones, smart phones, tablet computers, and mobile media playing devices.

The instant mood may be provided to the subject as a static image, and the instant mood may be provided to the subject as a dynamic, colored geometrical image capable of changing shape and/or color over time.

At least one embodiment may be summarized as a method of identifying an emotion state of a subject, including: inputting a signal that includes at least one type of biological information from a subject; computing, using a first microprocessor, at least one characteristic numerical value from the signal; computing, using second microprocessor, an emotion factor from the at least one characteristic numerical value; comparing the emotion factor with at least one entry of a predefined decision means to get a proposed emotion state; providing the proposed emotion state to the subject; querying the subject as to the correctness of the proposed emotion state; receiving feedback information from the subject concerning the correctness of the proposed emotion state; and using the feedback information to refine the decision means to reflect the correctness of the proposed emotion state to the subject.

The first microprocessor and the second microprocessor may be the same microprocessor.

The at least one type of biological information includes voice information, and the voice information may be input using a personal electronic device including the first microprocessor.

The personal electronic device may be selected from the group consisting of mobile phones, smart phones, tablet computers, and mobile media playing devices.

The second microprocessor may be located remotely from the subject, and the second microprocessor may be located in a cloud computing infrastructure.

The voice information may undergo noise cancellation.

The identified emotion state may be provided to the subject using a personal electronic device including the first microprocessor, and the personal electronic device may be selected from the group consisting of mobile phones, smart phones, tablet computers, and mobile media playing devices.

The identified emotion state may be provided to the subject as a static image, and the identified emotion state may be provided to the subject as a dynamic, colored image capable of changing shape and/or color over time.

At least one embodiment may be summarized as a system for identifying an instant mood of a subject, the system using any of the methods of identifying an instant mood of a subject of this disclosure.

At least one embodiment may be summarized as a system for identifying an emotion state of a subject, the system using any of the methods of identifying an emotion state of a subject of this disclosure.

At least one embodiment may be summarized as a method of refining a decision means, including: providing a stimulus expected to cause a predefined expected emotion state to a subject; allowing the subject to experience the stimulus for an amount of time equal to or greater than a predefined minimum time; receiving a signal that includes at least one type of biological information from the subject; computing at least one characteristic numerical value from the signal; and using the characteristic numerical value to refine a decision means to reflect a correlation between the predefined expected emotion state and the characteristic numerical value.

### BRIEF DESCRIPTION ON THE DRAWINGS

In the drawings, identical reference numbers identify similar elements or acts. The sizes and relative positions of elements in the drawings are not necessarily drawn to scale. For example, the shapes of various elements and angles may not be drawn to scale, and some of these elements may be arbitrarily enlarged and positioned to improve drawing legibility. Further, the particular shapes of the elements as drawn, are not intended to convey any information regarding the actual shape of the particular elements, and have been solely selected for ease of recognition in the drawings

FIG. 1 is a flowchart illustrating a method of identifying an instant mood of a subject.

FIG. 2 is a diagram illustrating a decision tree as an example of a predefined decision means.

FIG. 3 is a flowchart illustrating a method of identifying an instant mood of a subject.

FIG. 4 is a flowchart illustrating a method of identifying an instant mood of a subject, including query and feedback.

FIG. 5 is a diagram illustrating a subject inputting voice information via a smart phone.

FIG. 6 is a diagram illustrating output of instant moods via a smart phone.

FIG. 7 is a flowchart illustrating a method of identifying an emotion state of a subject, including query and feedback.

FIG. 8 is a flowchart illustrating a method of refining a decision means.

FIG. 9 is a diagram illustrating providing a stimulus expected to cause a predefined expected emotion state to a subject

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with mechanical couplings including, but not limited to, fasteners and/or housings have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment,” or “an embodiment,” or “in another embodiment” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an electrically powered device including “a power source” includes a single power source, or two or more power sources. It should also be noted that the term “or” is generally employed in its sense including “and/or” unless the content clearly dictates otherwise.

The headings provided herein are for convenience only and do not interpret the scope or meaning of the embodiments.

A human subject may desire to receive information concerning the subject's current emotional state, instant mood, or non-instant mood. Alternatively, an independent party may desire to receive information concerning a subject's current emotional state of mood, such as a physician or caregiver, a customer service worker, or an emergency services attendant. It may be beneficial to provide that information to the subject or the independent party using one or more physical characteristics of the subject at the time the information is provided. The one or more physical characteristics may include biological information from the subject.

As used herein, the term “emotion factor” means a numerical value capable of being related to at least one normal human emotion by referencing a decision means such as a lookup table, database, decision tree, or the like. An emotion factor may generally be found from biological information from a test subject.

As used herein, the term “emotion state” means at least one human emotion. An emotion state may be described verbally, in written language, visually, aurally, texturally, or via odor or smell.

As used herein, the term “non-instant mood” is equivalent to the simple term “mood” in general use in fields such as psychology. The qualifier “non-instant” has been chosen to differentiate this type of mood from an “instant” mood. Non-instant moods may exhibit a higher level of inertia and stability over a longer period of time, typically one or two days, compared to instant moods.

As used herein, the term “instant mood” means a mood state determined from an emotion factor representing one instant or short period of time, generally several seconds or less in length. An instant mood may be more variable in nature than a non-instant mood.

Note that although the terms instant mood and non-instant mood are defined for use throughout this disclosure, in general discussion the term mood may appear without any qualifiers. Such usages should generally be understood to mean non-instant mood.

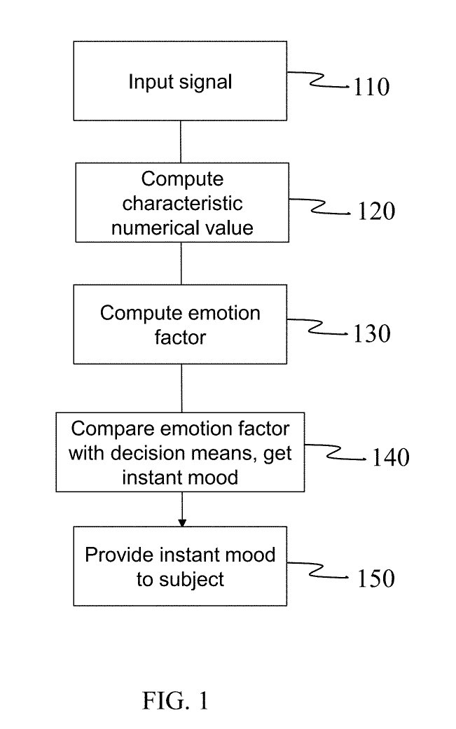

FIG. 1 is a flowchart illustrating a method of identifying an instant mood of a subject. Biological information from the subject may allow for identification of an instant mood. At 110, a signal including at least one type of biological information is input from a subject. A first microprocessor then uses the biological information signal to compute a characteristic numerical value at 120. The characteristic numerical value computed will generally differ depending upon the specific properties of the signal at the time the signal is input. The biological signal itself will tend to change over time depending upon what types of emotions and/or moods the subject is experiencing.

At 130, a second microprocessor then computes an emotion factor from the characteristic numerical value. The second microprocessor may be separated from the first microprocessor by any arbitrary distance. For example, the second microprocessor may be in a different room in the same building as the first microprocessor. The second microprocessor may also be in a location without easy physical access from the first microprocessor location, such as in a different building, which may be in a different town, city, or country. The second microprocessor may also be located in a cloud computing infrastructure. At times, the first microprocessor and the second microprocessor may be the same microprocessor.

The emotion factor is then compared with entries in a decision means at 140 to identify an instant mood. The decision means at 140 may employ any desired means, such as a decision tree, or a database having a multiple entries connecting specific emotion factors with instant moods. The decision means at 140 may also incorporate further computation or refining processes as long as the emotion factor provided leads to identification of an instant mood.

The instant mood identified is then provided to the subject at 150. Any method of providing information to the subject may be used, including text, sound, visual, smell, and tactile output. Instant moods may generally range along a continuous range from good to bad, and it may be preferable to use a method that allows the subject to understand the level of the mood, whether toward the good side or toward the bad side of the mood range. Specific numerical results may be provided if text or sound output is selected, while a more general mood level may be provided if terms such as great, very good, good, fair, not so good, under the weather, and the like are used. Alternatively, the strength of the output may be used to represent the mood level, such as sound volume, brightness of a visual display, or intensity of tactile stimulus such as vibration.

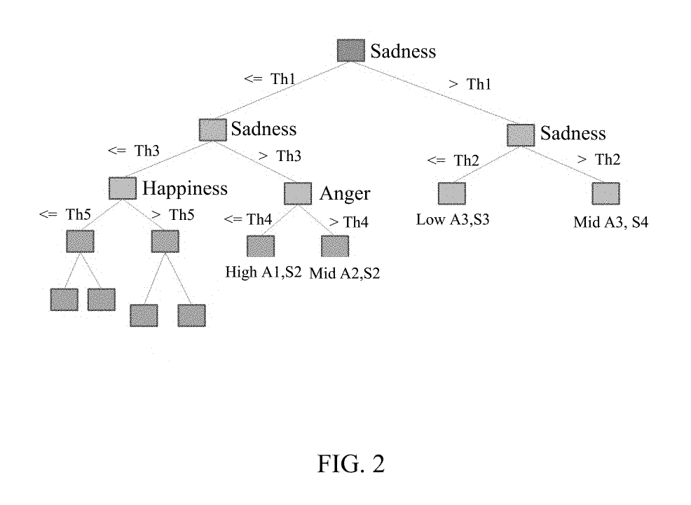

FIG. 2 is a diagram illustrating a representation of a partial decision tree as one non-limiting example of a predefined decision means used at 140. The decision tree may be used to examine the emotion factor and the distinct human emotions it represents in order to determine an instant mood. When the contribution of sadness to the instant mood is investigated, a numerical value of the emotion factor representing sadness may be compared to threshold values such as Th1, Th2, Th3, Th4, and Th5. The contribution of sadness to an instant mood may depend upon whether the numerical value is greater than, or equal to or less than, an individual threshold. Proceeding through the decision tree from the top may require comparisons with anger and/or happiness to accurately identify an instant mood. Once a final node of the decision tree is reached, resulting in an identification of High A1,S2, or Mid A2,S2, or the like, the identification may be converted a value that represents how much an instant mood is good or bad.

The threshold values Th1, Th2, Th3, Th4, and Th5 may initially be selected and averaging data acquired by testing a large population of subjects. The threshold values may not be optimal for a specific subject using the method of identifying an instant mood, however, and by adding optional querying steps to the method, the threshold values may be tuned or refined to an individual subject as the subject repeatedly uses the method. Alternatively, the subject may be asked to experience a certain stimulus intended to provoke a specific emotional response, after which biological information from the subject may be used to tune or refine the threshold values.

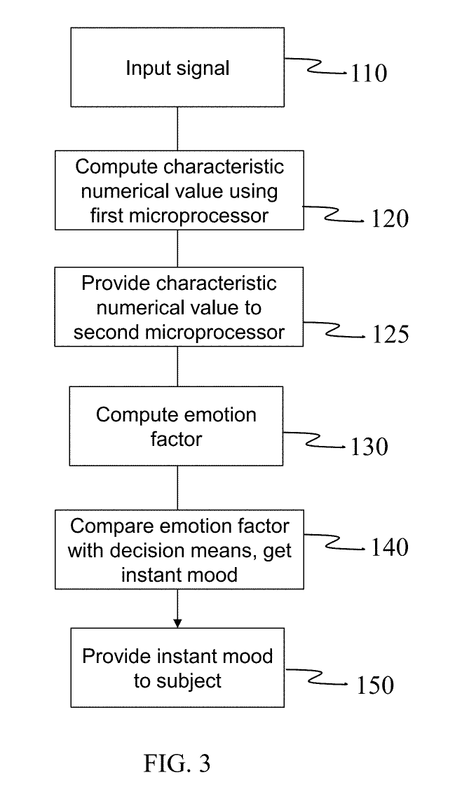

FIG. 3 is a flowchart illustrating a method of identifying an instant mood of a subject. In addition to all of the processes illustrated in FIG. 1, at 125 the characteristic numerical value computed at 120 is provided to the second microprocessor. The method of FIG. 3 may be employed when the first microprocessor is not the same as the second microprocessor. There are no limitations on how the characteristic value is provided. For example, the characteristic value may be provided via a network connection if the first and the second microprocessor are connected through a local area network, through the Internet, or through a mobile telephone network. The connection may also be a direct connection between two computing devices, one including the first microprocessor and the other one including the second microprocessor. A direct connection may be a physical connection using a wire or other electrically or optically conductive medium, or may be through a wireless connection such as WiFi.

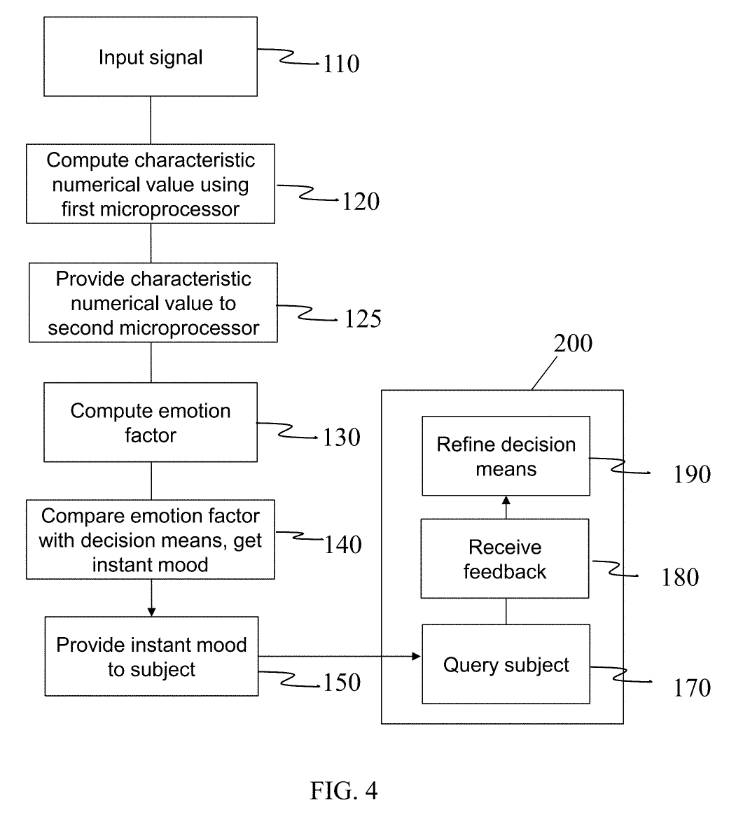

FIG. 4 is a flowchart illustrating a method of identifying an instant mood of a subject, including query and feedback. A predefined decision means, such as the decision tree 700 of FIG. 2, may initially contain computational or comparative rules, associative values, or other means useful for determining an appropriate instant mood for the emotion factor found at 130. The rules, values or the like may comprise results found by carrying out a comprehensive study of the general human population. Such results will skew toward the average. While they may be considered a viable starting point for determining an instant mood for a subject, it may be desirable to have a way of tuning or refining the decision means to make it more suited for a particular subject.

One method of refining the decision means is to gather feedback information from the subject after providing an instant mood thereto. FIG. 4 illustrates processes at 200 to implement information gathering. After an instant mood is provided to the subject at 150, the subject is then queried at 170 as to the correctness of the instant mood provided. The subject may reflect on how he or she actually feels compared the instant mood provided, and then provide feedback at 180. The subject may not be able to determine with precision just how accurate the instant mood is, but will likely be able to answer whether the instant mood is mostly correct, or shows a mood that is clearly better than (or worse than) the actual mood experienced by the subject. The feedback is then used at 190 to refine and made adjustments to the decision means, thus personalizing it to the individual subject.

The queries 170 may be made at regular intervals, and may also be made at random intervals. It may be preferable that the subject be able to activate or deactivate the query process at will. In addition, it may prove desirable that querying be implemented at certain specific times of the day, over a period of several days, when the subject is likely to be in a similar psychological state, such as in the morning just before or after breakfast. It may also be beneficial to query the subject after an especially positive or negative experience because such experiences may help define bounds of high and low instant moods.

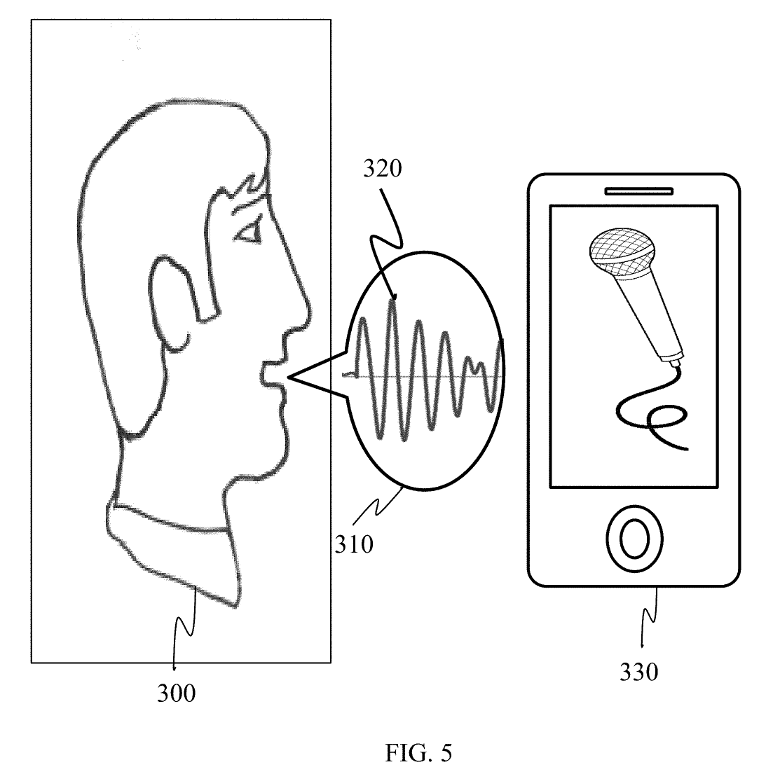

FIG. 5 is a diagram illustrating a subject inputting voice information via a smart phone. To make it convenient for a subject to use the method of identifying an instant mood, it may be helpful to provide a way for the subject to input biological information using a personal electronic device that is readily at hand. All types of personal electronic devices may be provided with the method of identifying an instant mood via preloaded programming, downloadable or otherwise installable application. Mobile phones, smart phones, table computers, and mobile media playing devices may be preferably used.

A subject 300 may input a signal 320 containing biological information into a personal electronic device 330. Biological information may include voice information, for example voice information 320, spoken into a microphone or other audio input means. Biological information may also include temperature information garnered from the subject using at least one temperature sensor, and may also include visual information such as facial expressions input using a static or dynamic video camera.

A fixed amount of biological information may be used for computing the characteristic value at 120, for example over a predetermined amount of time, such as five seconds. Alternatively, if the biological information is voice information, a single unit of speech having a loudness exceeding a predefined level over a fixed period of time. For example, a single unit of speech may consist of several words at the beginning of a sentence, or a simple phrase or interjection. With voice information, properties such as intensity, cadence, inflection, and the like may be measured from the signal 320 and included in the characteristic numerical value computation at 120.

Other biological information may also be employed, for instance moisture levels corresponding to perspiration levels of the subject and input via sensors placed at one or more locations on the subject's body. Heart rate, respiration rate, internal body temperature, skin temperature, blood pressure, and other physical characteristics may also be used to obtain the signal 320 containing biological information by using one or more suitable sensors capable of measuring the desired characteristic. Multiple types of biological information may also be input in parallel or in sequence in order to increase the accuracy of characteristic value computations.

A smart phone may be preferably chosen as the personal electronic device 330 when the signal 320 contains voice information. Mobile telephones, including smart phones, necessarily have a voice input means such as a built in microphone that readily allows for sound input of the signal 320 when spoken as voice information 310.

Using a smart phone as the personal electronic device 330 allows the characteristic numerical value to be provided to the second microprocessor in 125 via WiFi over a local area network when the second microprocessor is within the same building or organization, over the Internet when the second microprocessor is located at a different location, including in a cloud computing infrastructure, and over a voice and data carriers network when the smart phone subscribed to that network. The personal electronic device 330 may employ noise reduction to help reduce or eliminate ambient sounds present when the subject inputs voice information. The noise reduction may be included in the normal input algorithm that the smart phone uses, and may also include supplemental noise reduction carried out after the signal is input at 110.

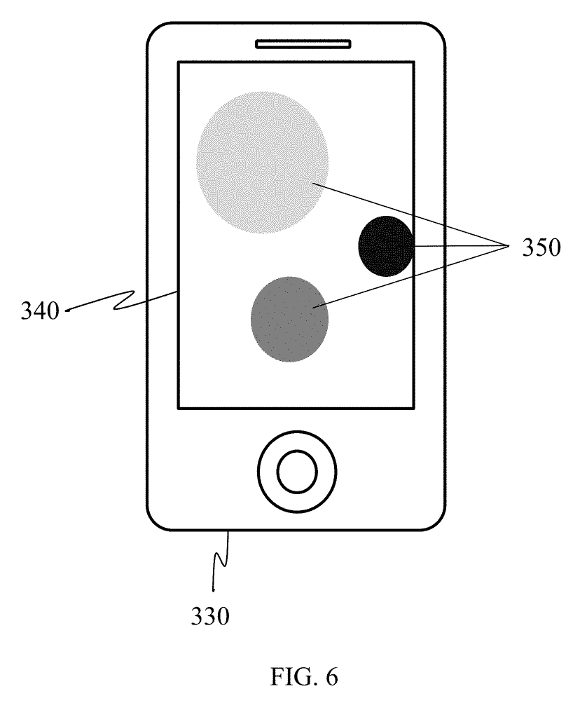

FIG. 6 is a diagram illustrating output of instant moods via a smart phone. The instant mood identified at 140 and output to the subject at 150 may preferably be displayed using a smart phone as the personal electronic device 330. An easily viewable screen 340 allows the instant mood to be output as an image 350. The image 350 may be a static image, displayed for a predetermined amount of time, and may also be a dynamic image capable of moving from one location to another within the screen and/or changing size or shape over a predetermined amount of time. The image 350 may be displayed in color, and multiple images 350 may be displayed together within the predetermined amount of time, each of the multiple images 350 representing a distinct instant mood. To clarify the distinct instant moods, each of the multiple images 350 may use a different color or shape.

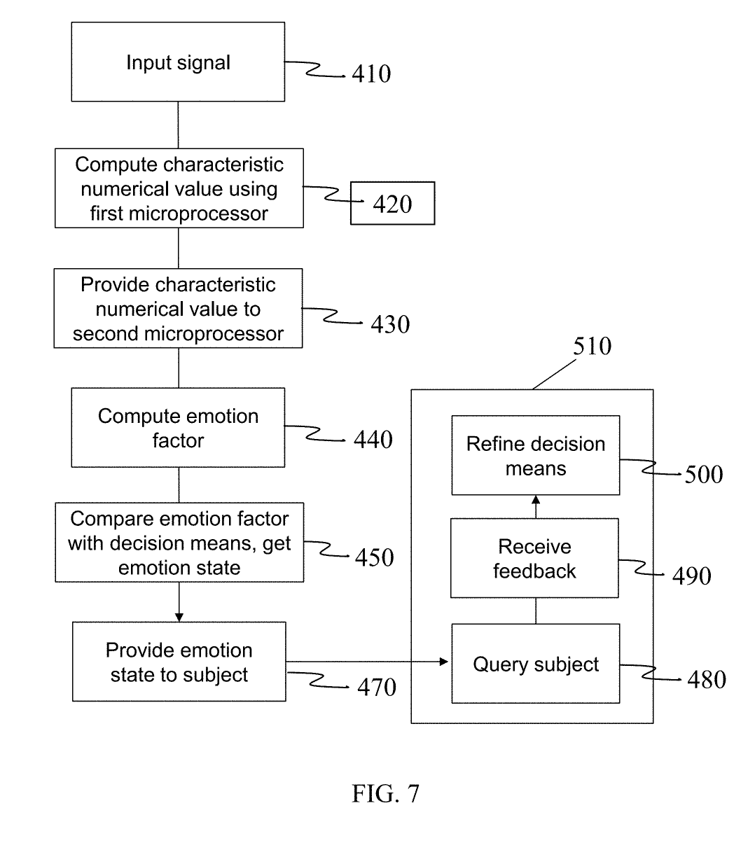

FIG. 7 is a flowchart illustrating a method of identifying an emotion state of a subject, including query and feedback. In addition to instant mood identification, it may benefit a subject to understand the basic emotions that are at the root of the instant mood. At least three distinct human emotions may be used in identifying an instant mood, and the three distinct human emotions may include anger, sadness, and happiness. Information on levels of these emotions may be beneficially provided to the subject in addition to, or instead of, an identified instant mood.

Furthermore, it may be desirable to receive feedback from the subject regarding the correctness of the three emotions of anger, sadness, and happiness in order to more accurately compute an emotion factor from a characteristic numerical value computed based on an input signal that includes at least one type of biological information. Similar to tuning or refining a decision means capable of providing an instant mood from an emotion factor, tuning or refining emotion factor computations may increase the usefulness and effectiveness, as well as increase accuracy and personalize, computation of an emotion factor targeted to a specific subject.

At 410, a signal including at least one type of biological information is input from a subject. A first microprocessor then uses the biological information signal to compute a characteristic numerical value at 420. The characteristic numerical value computed will generally differ depending upon the specific properties of the signal at the time the signal is input. The biological signal itself will tend to change over time depending upon what types of emotions and/or moods the subject is experiencing.

At 430, the characteristic numerical value computed at 420 is provided to a second microprocessor, which then computes an emotion factor from the characteristic numerical value at 440. The second microprocessor may be separated from the first microprocessor by any arbitrary distance. For example, the second microprocessor may be in a different room in the same building as the first microprocessor. The second microprocessor may also be in a location without easy physical access from the first microprocessor location, such as in a different building, which may be in a different town, city, or country. The second microprocessor may also be located in a cloud computing infrastructure. At times, the first microprocessor and the second microprocessor may be the same microprocessor.

The emotion factor is then compared with entries in a decision means at 450 to identify at least one emotion state. The decision means used in the process at 450 generally differs from the decision means used in the process at 140 in that in that at least one distinct human emotion is identified from the emotion factor. Three distinct human emotions are preferably identified, including anger, sadness, and happiness. The decision means at 450 may employ any desired means, such as a decision tree, or a database having a multiple entries connecting specific emotion factors with distinct human emotions.

The at least one emotion state is then provided to the subject at 470. Any method of providing information to the subject may be used, including text, sound, visual, smell, and tactile output. Emotion states for individual human emotions may generally differ along a continuous range from weak to strong, and it may be preferable to use a method that allows the subject to understand the level of individual human emotion. Specific numerical results may be provided if text or sound output is selected, while a more general level may be provided if terms such as very low, low, below average, average, above average, high, and very high or the like are used. Alternatively, the strength of the output may be used to represent the individual human emotion level, such as sound volume, brightness of a visual display, or intensity of tactile stimulus such as vibration.

When the emotion state includes more than one distinct human emotion, output may be made for each emotion simultaneously when using a visual method, and may be made in a predefined sequence if using another method that does not lend itself to conveying more than one level of information simultaneously.

A predefined decision means, such as the decision tree 700 of FIG. 2, may initially contain computational or comparative rules, associative values, or other means useful for determining an appropriate instant mood for the emotion factor found at 130. The rules, values or the like may comprise results found by carrying out a comprehensive study of the general human population. Such results will skew toward the average. While they may be considered a viable starting point for determining an instant mood for a subject, it may be desirable to have a way of tuning or refining the decision means to make it more suited to a particular subject.

After an emotion state including at least one, and preferably at least three, distinct human emotions is provided to the subject at 470, the subject is then queried at 480 as to the correctness of the emotion state provided. The subject may reflect on how he or she actually feels compared the emotion state provided, and then provide feedback at 490. The subject may not be able to determine with precision just how accurate the emotion state is, but will likely be able to answer whether the emotion state is mostly correct, or shows an emotion level that is clearly stronger than (or weaker than) the actual emotion state experienced by the subject. The feedback is then used at 510 to refine and made adjustments to the process for computing an emotion factor from a characteristic numerical value. If more the emotion state provided includes more than one distinct human emotion, the processes 480 and 490 may be repeated once for each emotion provided.

The queries at 480 may be made at regular intervals, and may also be made at random intervals. It may be preferable that the subject be able to activate or deactivate the query process at will. In addition, it may prove desirable that querying be implemented at certain specific times of the day, over a period of several days, when the subject is likely to be in a similar psychological state, such as in the morning just before or after breakfast. It may also be beneficial to query the subject after an especially positive or negative experience because such experiences may help define bounds of high and low emotion states.

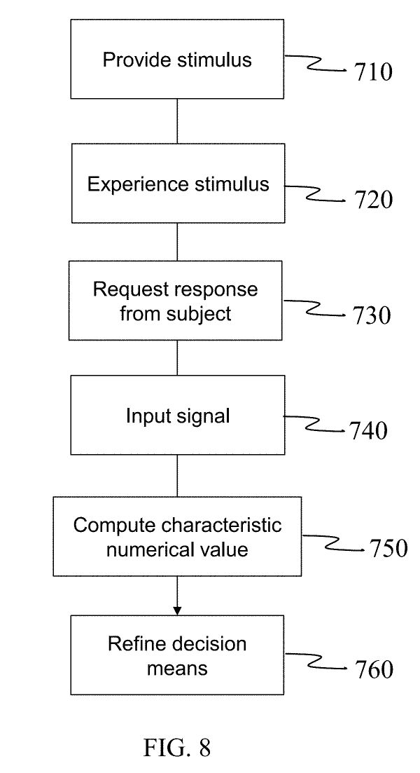

FIG. 8 is a flowchart illustrating a method of refining a decision means. It may be desirable to refine or tune a decision means originally constructed based on average data covering a large population of test subjects to more accurately reflect nuances unique to a particular subject. The decision means used in this disclosure to determine an instant mood from an emotion factor may be refined using this method, as may other decision means not related to instant mood and/or emotion state identification.

A stimulus may be provided to a subject at 710. The stimulus may be preferably chosen to provoke a similar response in the majority of the general populous, such as predominantly anger or happiness. The subject is allowed to experience the stimulus at 720 for at least a predefined amount of time. The amount of time is preferably set long enough to allow the subject to fully experience the stimulus and generate an emotional response, but not too long such that the emotional response may become muted.

At 730 the subject may be optionally requested to provide a response, generally by spoken, if biological information is to be gathered from a signal corresponding to speech. A signal including biological information may then be input at 740 from the subject. If biological information other than speech, for example facial expressions, body or skin temperature, skin inductance, or the like is used, then the request at 730 may be skipped and the information may be input directly from one or more sensors capable of generating a signal from the desired biological information type.

A characteristic numerical value is then computed at 750, and is used in refining the decision means at 760 to be more suitable for the subject. Note that the method of refining a decision means does not depend upon any specific determination of emotion state or instant mood provided by the subject. Instead, the subject is provided with a stimulus constructed to provoke a certain emotional response, and an uncontrolled or unconscious biological response from the subject is measured.

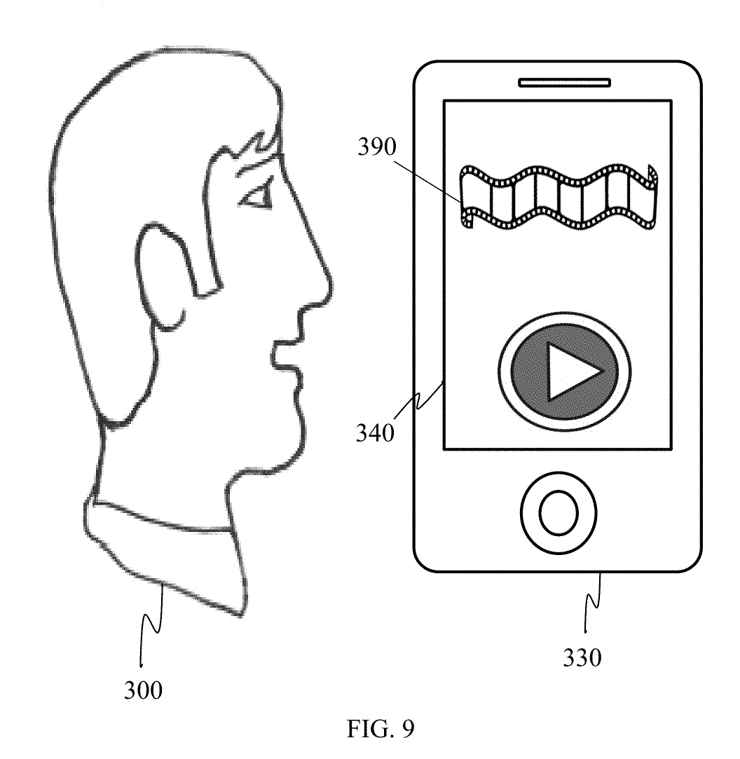

FIG. 9 is a diagram illustrating providing a stimulus expected to cause a predefined expected emotion state to a subject. The smart phone 330 may be advantageously used to provide the stimulus to the subject due to its portability as well as its ability to display static images, dynamic images and videos, and to play sounds such as voices, conversations, and music. A video clip 390 may be shown to the subject 300 as the stimulus, for example. Use of a smart phone also allows an optional request to be made to the subject, and allows the subject to input a response by voice input through the smart phone microphone, thus reducing the number of physical devices or implements needed for the subject during the method of refining a decision means.

The various embodiments described above can be combined to provide further embodiments. All of the U.S. patents, U.S. patent application publications, U.S. patent applications, foreign patents, foreign patent applications and non-patent publications referred to in this specification are incorporated herein by reference, in their entirety.

Aspects of the various embodiments can be modified, if necessary, to employ devices, apparatuses, and concepts of the various patents, applications and publications to provide yet further embodiments, including those patents and applications identified herein.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

I/We claim:

1. A method of providing an instant mood to a subject, comprising:
inputting a signal that includes at least one type of biological information from a subject;
computing, using a first microprocessor, at least one characteristic numerical value from the signal;
computing, using a second microprocessor, at least one emotion factor from the at least one characteristic numerical value;
comparing the emotion factor with at least one entry of a predefined decision means;
identifying an instant mood from results of comparing the emotion factor and the at least one entry of the predefined decision means; and
providing the instant mood to the subject.

2. The method of providing an instant mood to a subject according to claim 1, wherein the first microprocessor and the second microprocessor are the same microprocessor.

3. The method of providing an instant mood to a subject according to claim 1, further comprising:
providing the at least one characteristic numerical value to the second microprocessor.

4. The method of providing an instant mood to a subject according to claim 3, wherein the predefined decision means is refined for the subject being tested.

5. The method of providing an instant mood to a subject according to claim 3, further comprising:
querying the subject as to the correctness of the provided instant mood;
receiving feedback information from the subject concerning the correctness of the provided instant mood; and
using the feedback information to refine the decision means to reflect the feedback information from the subject.

6. The method of providing an instant mood to a subject according to claim 3, wherein the at least one type of biological information includes voice information.

7. The method of providing an instant mood to a subject according to claim 6, wherein the voice information comprises a unit of spoken speech.

8. The method of providing an instant mood to a subject according to claim 7, wherein the emotion factor represents at least three distinct human emotions.

9. The method of providing an instant mood to a subject according to claim 8, wherein the three distinct human emotions are anger, sadness, and happiness.

10. The method of providing an instant mood to a subject according to claim 8, further comprising storing the identified instant mood in a non-volatile storing means.

11. The method of providing an instant mood to a subject according to claim 10, wherein the instant mood is computationally weighted against at least one previously identified instant mood stored in the non-volatile storing means.

12. The method of providing an instant mood to a subject according to claim 6, wherein the voice information is input using a personal electronic device including the first microprocessor.

13. The method of providing an instant mood to a subject according to claim 12, wherein the personal electronic device is selected from the group consisting of mobile phones, smart phones, tablet computers, and mobile media playing devices.

14. The method of providing an instant mood to a subject according to claim 13, wherein the second microprocessor is located remotely from the subject.

15. The method of providing an instant mood to a subject according to claim 13, wherein at least one of the second microprocessor and the non-volatile storing means is located in a cloud computing infrastructure.

16. The method of providing an instant mood to a subject according to claim 13, wherein the voice information undergoes noise cancellation.

17. The method of providing an instant mood to a subject according to claim 6, wherein the instant mood is provided to the subject using a personal electronic device including the first microprocessor.

18. The method of providing an instant mood to a subject according to claim 17, wherein the personal electronic device is selected from the group consisting of mobile phones, smart phones, tablet computers, and mobile media playing devices.

19. The method of providing an instant mood to a subject according to claim 18, wherein the instant mood is provided to the subject as a static image.

20. The method of providing an instant mood to a subject according to claim 18, wherein the instant mood is provided to the subject as a dynamic, colored geometrical image capable of changing shape and/or color over time.

21. A method of providing an emotion state to a subject, comprising:
inputting a signal that includes at least one type of biological information from a subject;
computing, using a first microprocessor, at least one characteristic numerical value from the signal;
computing, using second microprocessor, an emotion factor from the at least one characteristic numerical value;
comparing the emotion factor with at least one entry of a predefined decision means;
identifying a proposed emotion state from results of comparing the emotion factor and the at least one entry of the predefined decision means; and
providing the proposed emotion state to the subject;
querying the subject as to the correctness of the proposed emotion state;
receiving feedback information from the subject concerning the correctness of the proposed emotion state; and
using the feedback information to refine the decision means to reflect the correctness of the proposed emotion state to the subject.

22. The method of providing an emotion state to a subject according to claim 21, wherein the first microprocessor and the second microprocessor are the same microprocessor.

23. The method of providing an emotion state to a subject according to claim 21, wherein the at least one type of biological information includes voice information.

24. The method of providing an emotion state to a subject according to claim 23, wherein the voice information is input using a personal electronic device including the first microprocessor.

25. The method of providing an emotion state to a subject according to claim 24, wherein the personal electronic device is selected from the group consisting of mobile phones, smart phones, tablet computers, and mobile media playing devices.

26. The method of providing an emotion state to a subject according to claim 25, wherein the second microprocessor is located remotely from the subject.

27. The method of providing an emotion state to a subject according to claim 25, wherein the second microprocessor is located in a cloud computing infrastructure.

28. The method of providing an emotion state to a subject according to claim 25, wherein the voice information undergoes noise cancellation.

29. The method of providing an emotion state to a subject according to claim 23, wherein the identified emotion state is provided to the subject using a personal electronic device including the first microprocessor.

30. The method of providing an emotion state to a subject according to claim 29, wherein the personal electronic device is selected from the group consisting of mobile phones, smart phones, tablet computers, and mobile media playing devices.

31. The method of providing an emotion state to a subject according to claim 30, wherein the identified emotion state is provided to the subject as a static image.

32. The method of providing an emotion state to a subject according to claim 30, wherein the identified emotion state is provided to the subject as a dynamic, colored image capable of changing shape and/or color over time.

33. A system for providing an instant mood to a subject, the system using the method of providing an instant mood of a subject according to claim 3.

34. A system for providing an emotion state to a subject, the system using the method of providing an emotion state to a subject according to claim 21.
